(19) United States
(12) Patent Application Publication
(10) Pub. No.: US 2009/0112333 A1
(43) Pub. Date: Apr. 30, 2009

(54) SMART INFUSION PUMP

(76) Inventor: ANIL K. SAHAI, Webster City, IA (US)

Correspondence Address: MCKEE, VOORHEES & SEASE, P.L.C., 801 GRAND AVENUE, SUITE 3200, DES MOINES, IA, 50309-2721, US

(21) Appl. No.: 11/926,325

(22) Filed: Oct. 29, 2007

(51) Int. Cl. G05B 19/048; A61M 31/00; A61M 5/142

(52) U.S. Cl. 700/3; 604/246; 604/67

(57) ABSTRACT

An apparatus for delivering a fluid to a patient is provided. The apparatus includes an infusion pump for delivering the fluid, a control unit electrically connected to the infusion pump, a display operatively connected to the control unit, a communications interface operatively connected to the control unit and adapted for wireless communication, and an RFID reader operatively connected to the control unit. The control unit is configured for reading an RFID tag associated with an infusate container containing the fluid and reading an RFID tag associated with the patient.

### FIELD OF THE INVENTION

The present invention relates to infusion pumps. More particularly, the present invention relates to an intelligent infusion pump.

### BACKGROUND OF THE INVENTION

Various types of infusion pumps are commercially available for infusing fluids, medication or nutrients into a patient. Typically a motor or a peristaltic pump is used to perform the pumping and it is common that the procedure is performed under electronic control. Yet, despite advancements made with respect to infusion pumps, multitudes of problems such as wrong medications and rate of infusion, etc. still do exist. That may lead to longtime mortality and morbidity.

Therefore, it is a primary object, feature, or advantage of the present invention to improve over the state of the art by providing an intelligent infusion pump.

### BRIEF SUMMARY OF THE INVENTION

According to one aspect of the present invention, an apparatus for delivering a fluid to a patient is provided. The apparatus includes an infusion pump for delivering the fluid, a control unit electrically connected to the infusion pump, a display operatively connected to the control unit, a communications interface operatively connected to the control unit and adapted for wireless communication, and an RFID reader operatively connected to the control unit. The control unit is configured for reading an RFID tag associated with an infusate container containing the fluid and reading an RFID tag associated with the patient.

According to another aspect of the present invention a system is provided. The system includes an apparatus for delivering a fluid to a patient, comprising: (a) an infusion pump for delivering the fluid, (b) a control unit electrically connected to the infusion pump, (c) a display operatively connected to the control unit, (d) a communications interface operatively connected to the control unit and adapted for wireless communication, (e) an RFID reader operatively connected to the control unit. The system also includes an infusate container comprising a vessel, an infusate within the vessel, and an RFID tag operatively connected to the vessel. The system also includes a patient wristband comprising a wristband and an RFID tag operatively connected to the wristband.

According to another aspect of the present invention, a method for delivering infusate to a patient is provided. The method includes reading an RFID tag associated with an infusate container to provide infusate container data, wherein the infusate container data includes a first patient identifier and infusion pump settings, reading an RFID tag associated with a patient to provide patient data, wherein the patient data includes a second patient identifier. The method further includes determining if the first patient identifier corresponds with the second patient identifier. If the first patient identifier corresponds with the second patient identifier, setting an infusion pump using the infusion pump settings.

### BRIEF DESCRIPTION OF THE DRAWINGS

FIG. 1 is a block diagram illustrating one embodiment of a system of the present invention.

FIG. 2 is a block diagram illustrating an infusion pump of FIG. 1.

FIG. 3 is a pictorial representation of one embodiment of the infusion pump of FIG. 1 and FIG. 2.

FIG. 4 is block diagram illustrating the infusion pump of FIG. 1-3 in communication with other devices.

### DETAILED DESCRIPTION

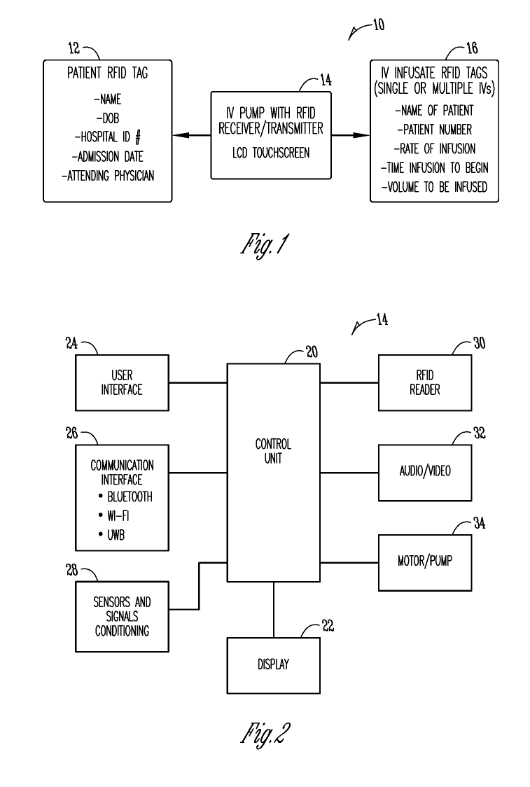

The present invention provides for an infusion pump methods and systems related to the infusion pump. FIG. 1 illustrates one embodiment of a system 10 according to the present invention. A patient RFID tag 12 is shown. The patient RFID tag 12 includes patient data such as, without limitation, patient name, patient date of birth, a hospital identification number associated with the patient, an admission date for the patient, and the name of an attending physician. Of course, the patient RFID tag 12 may also include other information. The patient RFID tag 12 is read by an infusion pump 14. The infusion pump 14 includes an RFID reader or interrogator which may read the patient RFID tag 12. The infusion pump 14 also preferably includes an LCD touchscreen display. The infusion pump 14 may also read RFID tags associated with infusate containers 16. The RFID tags on the infusate containers may include information such as the name of a patient, the patient number or hospital number associated with the patient, the rate of infusion, the time the infusion is to begin, the volume to be infused, the type of infusate, or other information.

Thus, in additional to performing functions conventionally associated with an infusion pump, the infusion pump also is able to read RFID tags associated with a patient as well as RFID tags associated with infusate containers. The infusion pump may use this information for a variety of reasons including to improve safety, automate processes, and improve patient care.

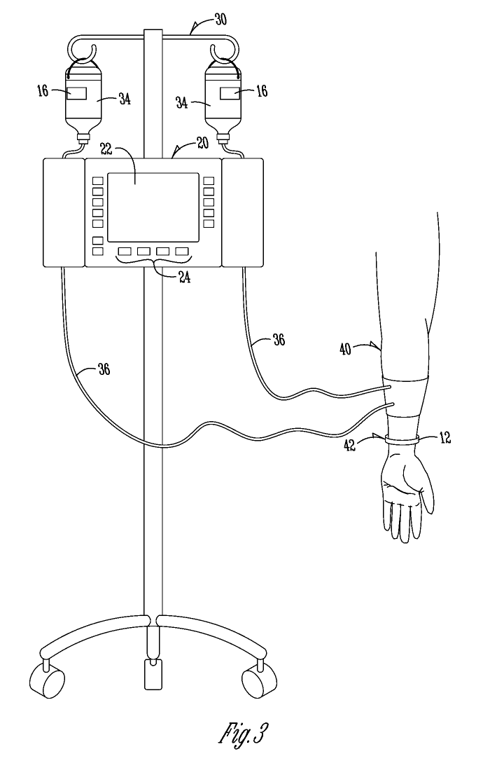

FIG. 2 is a block diagram illustrating one embodiment of the infusion pump 14 shown in FIG. 1. The infusion pump 14 includes a motor and/or pump 24 such as conventionally associated with an infusion pump. The infusion pump 14 also includes a control unit 20. The control unit 20 may include an intelligent control such as a micro controller, a processor, a computer, an application specific integrated circuit, or other type of intelligent control. The control unit 20 is electrically connected to a display 22. The display 22 may be a touch panel display. The control unit 20 is also electrically connected to a user interface 24 which may be associated with the display 22, may include buttons, switches, knobs, or other types of manual inputs separate from the display 22. The user interface 24 may also include additional displays or indicators.

The control unit 20 is also electrically connected to a communication interface 20. The communication interface 20 may be of any number of types, including, without limitation, Bluetooth, Wi-Fi, or UWB. The communication interface 20 allows the infusion pump 14 to communicate with other devices such as, but not limited to thermometers, automatic blood pressure and patient weight machines, oxygen saturation sensors as well as to computers or other devices at the patient's bedside.

The control unit 20 is also electrically connected to sensors 28 and any appropriate signal conditioning associated with the sensors. The present invention contemplates that any number of sensors and any number of types of sensors may be used, including those associated with the infusion process. For example the sensors 28 may provide for sensing the amount of air in a given time duration (such as each minute). The sensors 28 may providing for sensing the bolus of air. The sensors 28 may provide for sensing the pressure in tubing for use in detecting occlusion. In addition, the sensors 28 may include sensors which assist in monitoring and controlling the rate of an infusion.

The infusion pump 14 also includes an RFID reader 30 or RFID interrogator. The RFID reader 30 may be used to read RFID tags associated with patients or RFID tags associated with infusate containers, or other RFID tags. The control unit 20 is programmed or otherwise configured to use patient data from an RFID tag associated with a patient as well as infusate data associated with an RFID tag associated with an infusate container in order to monitor, control and safeguard the infusion process. The present invention contemplates that this information may be used in numerous ways and is not limited to the specific examples discussed.

A first example is when the control unit 20 is programmed or otherwise configured to verify the patient name associated with the patient RFID tag with the patient name associated with an infusate container. The control unit 20 does not permit the motor/pump 34 to operate unless the infusate container has been designated for use with the particular patient. Instead of patient name, a patient number or hospital identification number or other type of identifier may be used. Prior to the infusate being delivered to the patient, the RFID tag is written to include the patient name.

Another example is that the RFID tag may include information regarding the infusion process such as the rate of infusion, the time for infusion to begin, or the volume to be infused. This information may be written on an RFID tag as per an attending physician's instructions. The infusion pump 14 reads this information from the RFID tag using the RFID reader 30 and uses it to set or control infusion pump settings as opposed to relying on manually setting the infusion pump. Prior to the infusion pump 14 being activated, a nurse may confirm the settings and prior to initiating the infusion process.

Yet another example is that the RFID tag may include information regarding set limits regarding the amount of air. The amount of air in one minute or bolus of air may be sensed. The RFID tag may include set limits upon which an alarm would be triggered.

The present invention further contemplates that after an infusate is prescribed, this information is entered into a computer operatively connected to an RFID writer and that prior to an RFID tag associated with an infusate container being written or rewritten, electronic records associated with the patient may be automatically accessed and other comparisons made.

Returning to FIG. 2, audio/video alerts 32 may be electrically connected to the control unit. The audio/video device may include speakers, lights or other indicators in addition to the user interface 24 or the display 22. The audio/video device 32 may be used as alarms or to provide voice instructions. For example, if during the monitoring of the infusion process one or more of the sensors 28 indicate there is an occlusion, the control unit 20 may activate voice instructions for the patient to follow. For example, the instructions may be for the patient to “Please straighten your arm or wrist.” Such a simple instruction, once followed can eliminate the occlusion. The instruction may be repeated one or more times and if the sensed condition, such as the sensing of an occlusion, is still present then a nurse may be notified through communication with a nursing station or otherwise.

The audio/video alerts 32 may be used for alarm purposes, such as if the amount of air sensed in a given time period exceeds a set limit, or if pressure sensed in the tubing exceeds a particular pressure setting. In addition, or alternatively, alarms may be communicated directly to a nurse's station.

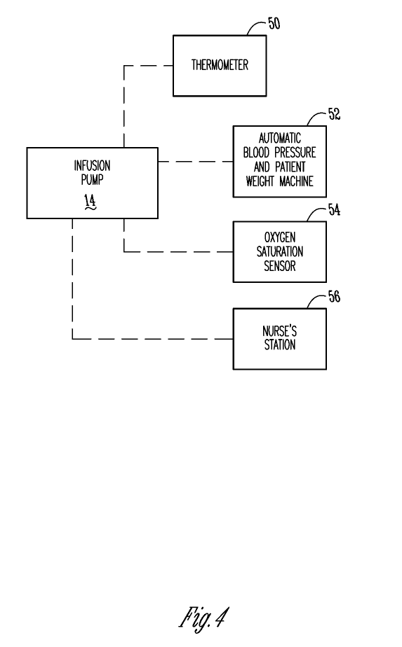

FIG. 3 provides a pictorial representation of one embodiment of the present invention. An infusion pump 20 is shown which includes a display 20 which is preferably a touch screen display as well as various manual inputs which may provide a portion of a user interface 24. Two separate infusate containers 34 are shown which may include fluids, medication or nutrients prepared for infusion into a patient 40. Each of the infusate containers 34 includes an RFID tag 16. Tubing 36 extends from the infusion pump 20 to the patient 40. The patient 40 wears a wristband which includes an RFID tag 12.

FIG. 4 illustrates an infusion pump 14. A thermometer 50, an automatic pressure and patient weight machine 52, an oxygen saturation sensor 54, and a nurse's station 56. As illustrated in FIG. 4, the infusion pump 14 may communicate with numerous other types of devices or sensors provided such devices are configured for communication with the infusion pump. In one embodiment each of these devices may include Bluetooth technology and the infusion pump 14 is similarly equipped with Bluetooth technology for wireless communication. Where additional information is available from other types of sensors, the present invention contemplates that the control unit of the infusion pump 14 may be programmed or otherwise configured to collect the information and use the information to determine alarm conditions, to modify the infusion process, or otherwise.

It is also to be understood that where RFID tag information is used to determine infusion pump settings, these settings may be modified or overridden, and the infusion pump may require that the settings be confirmed by a nurse before the infusion process is initiated.

Therefore, an intelligent infusion pump has been disclosed. The present invention contemplates numerous variations, alternatives, and options fall within the spirit and scope of the invention and the present invention is not to be limited to the specific embodiments described herein.

What is claimed is:

1. An apparatus for delivering a fluid to a patient, comprising:
an infusion pump for delivering the fluid;
a control unit electrically connected to the infusion pump;
a display operatively connected to the control unit;
a communications interface operatively connected to the control unit and adapted for wireless communication;
an RFID reader operatively connected to the control unit;
wherein the control unit configured for reading an RFID tag associated with an infusate container containing the fluid and reading an RFID tag associated with the patient.

2. The apparatus of claim 1 wherein the control unit is adapted to receive a first patient identifier from the RFID tag associated with the infusate container and a second patient identifier from the RFID tag associated with the patient and verify that the first patient identifier matches the second patient identifier prior to initiating the delivering of the fluid to the patient.

3. The apparatus of claim 2 wherein the control unit is adapted to receive infusion pump settings from the infusate container.

4. The apparatus of claim 1 further comprising at least one sensor operatively connected to the control unit for monitoring the delivering of the fluid to the patient.

5. The apparatus of claim 1 further comprising an audio device electrically connected to the control unit and wherein the control unit is adapted to provide voice messages to a patient using the audio device.

6. A system comprising:
an apparatus for delivering a fluid to a patient, comprising: (a) an infusion pump for delivering the fluid, (b) a control unit electrically connected to the infusion pump, (c) a display operatively connected to the control unit, (d) a communications interface operatively connected to the control unit and adapted for wireless communication, (e) an RFID reader operatively connected to the control unit;
an infusate container comprising a vessel, an infusate within the vessel, and an RFID tag operatively connected to the vessel;
a patient wristband comprising a wristband and an RFID tag operatively connected to the wristband.

7. The system of claim 6 wherein the control unit is adapted to receive a first patient identifier from the RFID tag associated with the infusate container and a second patient identifier from the RFID tag associated with the patient and verify that the first patient identifier matches the second patient identifier prior to initiating the delivering of the fluid to the patient.

8. The system of claim 7 wherein the control unit is adapted to receive infusion pump settings from the infusate container.

9. The system of claim 7 further comprising at least one sensor operatively connected to the control unit for monitoring the delivering of the fluid to the patient.

10. The system of claim 7 further comprising an audio device electrically connected to the control unit and wherein the control unit is adapted to provide voice messages to a patient using the audio device.

11. A method for delivering infusate to a patient, comprising:
reading an RFID tag associated with an infusate container to provide infusate container data, wherein the infusate container data includes a first patient identifier and infusion pump settings;
reading an RFID tag associated with a patient to provide patient data, wherein the patient data includes a second patient identifier;
determining if the first patient identifier corresponds with the second patient identifier;
if the first patient identifier corresponds with the second patient identifier, setting an infusion pump using the infusion pump settings.

12. The method of claim 11 further comprising receiving user input to confirm the infusion pump settings.

13. The method of claim 11 further comprising initiating the delivering of the infusate to the patient.

14. The method of claim 11 further comprising monitoring the delivering of the infusate to the patient.

15. The method of claim 11 further comprising monitoring the patient during the delivering of the infusate to the patient.

16. The method of claim 11 further comprising providing a voice instruction to the patient during the delivering of the infusate to the patient if a patient correctable condition is identified.
